(19) United States
(12) Patent Application Publication
(10) Pub. No.: US 2025/0186051 A1
(43) Pub. Date: Jun. 12, 2025

(54) GATE DRIVING CIRCUIT, DISPLAY DEVICE INCLUDING THE GATE DRIVING CIRCUIT AND ELECTRONIC DEVICE INCLUDING THE DISPLAY DEVICE

(71) Applicant: Samsung Display Co., Ltd., Yongin-Si (KR)

(72) Inventors: WONJUN LEE, Yongin-si (KR); SUNGMIN SON, Yongin-si (KR); Minji Kim, Yongin-si (KR); GEUMJU MOON, Yongin-si (KR)

(21) Appl. No.: 19/017,802

(22) Filed: Jan. 13, 2025

Related U.S. Application Data
(63) Continuation of application No. 17/157,379, filed on Jan. 25, 2021, now Pat. No. 12,193,676.
Application No. 17/157,379 is a continuation of application No. 15/676,869, filed on Aug. 14, 2017, now Pat. No. 10,898,201.
Application No. 15/676,869 is a continuation of application No. 14/586,110, filed on Apr. 23, 2015, now Pat. No. 9,730,700.
Application No. 14/586,110 is a continuation of application No. 13/243,941, filed on Sep. 23, 2011, now Pat. No. 8,926,682.
Application No. 13/243,941 is a division of application No. 12/429,474, filed on Apr. 24, 2009, now abandoned.
(60) Provisional application No. 61/048,038, filed on Apr. 25, 2008.

(30) Foreign Application Priority Data
Apr. 23, 2024 (KR) 10-2024-0053791

(51) Int. Cl. A61B 17/12; A61F 2/06; A61F 2/07; A61F 2/30; A61F 2/90; A61F 2/95; A61F 2/954; A61M 25/00; A61M 25/10

(52) U.S. Cl. CPC A61B 17/12118; A61B 17/12136; A61B 17/12186; A61B 17/12195; A61F 2/07; A61F 2/90; A61F 2/954; A61M 25/10182; A61M 25/10187; A61F 2002/067; A61F 2002/077; A61F 2002/30583; A61F 2002/30604; A61F 2002/9511; A61F 2210/0085; A61F 2230/0034; A61F 2250/0003; A61F 2250/006; A61M 25/007; A61M 25/1018; A61M 25/10188

(57) ABSTRACT

A gate driving circuit includes a normal output circuit configured to output a first gate signal at a first output node and a second gate signal at a second output node in response to a previous first gate signal and a first clock signal. The first gate signal and the second gate signal are opposite in phase to each other, and the first gate signal and the second gate signal are applied to different transistors included in a pixel circuit.

### PRIORITY STATEMENT

This application claims priority under 35 U.S.C. § 119 to Korean Patent Application No. 10-2024-0053791, filed on Apr. 23, 2024 in the Korean Intellectual Property Office KIPO, the contents of which are herein incorporated by reference in their entireties.

### BACKGROUND

### 1. Field

Embodiments of the present inventive concept relate to a gate driving circuit and a display device including the gate driving circuit. More particularly, embodiments of the present inventive concept relate to a gate driving circuit reducing a driving voltage of the gate driving circuit and a power consumption of a display device and the display device including the gate driving circuit.

### 2. Description of the Related Art

Generally, a display device includes a display panel and a display panel driving circuit. The display panel includes a plurality of gate lines, a plurality of data lines, a plurality of emission lines and a plurality of pixel circuits. The display panel driving circuit includes a gate driving circuit, a data driving circuit, an emission driving circuit and a driving control circuit. The gate driving circuit outputs gate signals to the gate lines. The data driving circuit outputs data voltages to the data lines. The emission driving circuit outputs emission signals to the emission lines. The driving control circuit controls the gate driving circuit, the data driving circuit and the emission driving circuit.

When transistors receiving a same gate signal in a pixel circuit are same type transistors (N-type transistors or P-type transistors), a driving voltage of the gate driving circuit applying the same gate signal to the pixel circuit may increase. When the driving voltage of the gate driving circuit increases, a power consumption of the display device may increase.

In addition, when another gate driving circuit is added to apply additional gate signals having phases opposite to phases of the gate signals, a dead space of the display device may increase.

### SUMMARY

Embodiments of the present inventive concept provide a gate driving circuit simultaneously applying two gate signals having opposite phases to a pixel circuit.

Embodiments of the present inventive concept provide a display device including the pixel circuit for reducing a driving voltage of the gate driving circuit.

Embodiments of the present inventive concept provide a electronic device including the display device.

In an embodiment of a gate driving circuit according to the present inventive concept, the gate driving circuit includes a normal output circuit configured to output a first gate signal at a first output node and a second gate signal at a second output node in response to a previous first gate signal and a first clock signal. The first gate signal and the second gate signal are opposite in phase to each other, and the first gate signal and the second gate signal are applied to different transistors included in a pixel circuit.

In an embodiment, the normal output circuit may include a ninth transistor including a control electrode configured to receive the first clock signal, a first electrode configured to receive the previous first gate signal and a second electrode connected to a first node, a tenth transistor including a control electrode connected to the first node, a first electrode configured to receive a first gate power supply voltage and a second electrode connected to the second output node, a eleventh transistor including a first electrode connected to the second output node and a second electrode configured to receive a second gate power supply voltage, a twelfth transistor including a control electrode configured to receive the second gate power supply voltage, a first electrode connected to the first node and a second electrode connected to a second node, a thirteenth transistor including a control electrode the second output node, a first electrode the first gate power supply voltage and a second electrode connected to the first output node, a fourteenth transistor including a control electrode connected to the second node, a first electrode connected to the first output node and a second electrode configured to receive the second gate power supply voltage, a first capacitor including a first electrode connected to the second node and a second electrode connected to the first output node and a second capacitor including a first electrode configured to receive the first gate power supply voltage and a second electrode connected to the second output node.

In an embodiment, the eleventh transistor may further include a control electrode connected to the second node.

In an embodiment, the eleventh transistor may further include a control electrode connected to the first node.

In an embodiment, the normal output circuit may further include a reset transistor including a control electrode configured to receive a reset signal, a first electrode connected to the second output node and a second electrode configured to receive the second gate power supply voltage.

In an embodiment of a gate driving circuit according to the present inventive concept, the gate driving circuit includes a normal output circuit configured to output a first gate signal at a first output node and controls a voltage of a second output node in response to a previous first gate signal and a first clock signal and an inverted output circuit configured to output a third gate signal at a third output node in response to a second clock signal and the voltage of the second output node. The first gate signal and the third gate signal are opposite in phase to each other, and the first gate signal and the third gate signal are applied to different transistors included in a pixel circuit.

In an embodiment, the inverted output circuit may include a fifteenth transistor including a control electrode configured to receive the second clock signal, a first electrode connected to the second output node and a second electrode connected to a buffer node, a sixteenth transistor including a control electrode configured to receive a second gate power supply voltage, a first electrode connected to the buffer node and a second electrode connected to a third node, a seventeenth transistor including a control electrode connected to the third node, a first electrode configured to receive the second gate power supply voltage and a second electrode connected to the third output node, a eighteenth transistor including a first electrode connected to the third output node and a second electrode configured to receive a first gate power supply voltage and a third capacitor including a first electrode connected to the third node and a second electrode connected to the third output node.

In an embodiment, the normal output circuit may include a ninth transistor including a control electrode configured to receive the first clock signal, a first electrode configured to receive the previous first gate signal and a second electrode connected to a first node, a tenth transistor including a control electrode connected to the first node, a first electrode configured to receive the first gate power supply voltage and a second electrode connected to the second output node, a eleventh transistor including a first electrode connected to the second output node and a second electrode configured to receive the second gate power supply voltage, a twelfth transistor including a control electrode configured to receive the second gate power supply voltage, a first electrode connected to the first node and a second electrode connected to a second node, a thirteenth transistor including a control electrode the second output node, a first electrode the first gate power supply voltage and a second electrode connected to the first output node, a fourteenth transistor including a control electrode connected to the second node, a first electrode connected to the first output node and a second electrode configured to receive the second gate power supply voltage, a first capacitor including a first electrode connected to the second node and a second electrode connected to the first output node and a second capacitor including a first electrode configured to receive the first gate power supply voltage and a second electrode connected to the second output node.

In an embodiment, the eighteenth transistor may further include a control electrode connected to the first output node.

In an embodiment, the eighteenth transistor may further include a control electrode connected to the second node.

In an embodiment, the normal output circuit may further include a reset transistor including a control electrode configured to receive a reset signal, a first electrode connected to the second output node and a second electrode configured to receive the second gate power supply voltage.

In an embodiment of a display device according to the present inventive concept, the display device includes a display panel including a pixel circuit, a gate driving circuit configured to provide a gate signal to the pixel circuit, a data driving circuit configured to provide a data voltage to the pixel circuit, and an emission driving circuit configured to provide an emission signal to the pixel circuit. The pixel circuit includes a light emitting element, a first transistor configured to provide a driving current to the light emitting element, a second transistor including a first electrode configured to receive a bias voltage and a second electrode connected to a first electrode of the first transistor and a third transistor including a first electrode connected a anode electrode of the light emitting element and a second electrode configured to receive a first initialization voltage. One of the second transistor and the third transistor is P-type transistor and an other one of the second transistor and the third transistor is N-type transistor.

In an embodiment, the second transistor may be P-type transistor and the third transistor is N-type transistor.

In an embodiment, the pixel circuit may further include a fourth transistor including a control electrode configured to receive a writing gate signal, a first electrode configured to receive the data voltage and a second electrode connected to the first electrode of the first transistor, a fifth transistor including a control electrode configured to receive a compensation gate signal, a first electrode connected to a control electrode of the first transistor and a second electrode connected to a second electrode of the first transistor, a sixth transistor including a control electrode configured to receive an initialization gate signal, a first electrode connected to the control electrode of the first transistor and a second electrode configured to receive a second initialization voltage, a seventh transistor including a control electrode configured to receive the emission signal, a first electrode configured to receive a first power voltage and a second electrode connected to the first electrode of the first transistor, a eighth transistor including a control electrode configured to receive the first power voltage, a first electrode connected to the second electrode of the first transistor and a second electrode connected to the first electrode of the third transistor and a storage capacitor including a first electrode configured to receive the first power voltage and a second electrode connected to the control electrode of the first transistor.

In an embodiment, the third transistor may further include a control electrode and a second control electrode connected to the control electrode of the third transistor, the fifth transistor may further include a second control electrode connected to the control electrode of the fifth transistor, and the sixth transistor may further include a second control electrode connected to the control electrode of the sixth transistor.

In an embodiment, the first transistor may further include a second control electrode configured to receive the first power voltage.

In an embodiment, the gate driving circuit may include a normal output circuit configured to output a first gate signal at a first output node and a second gate signal at a second output node in response to a previous first gate signal and a first clock signal. The first gate signal and the second gate signal may be opposite in phase to each other, and the first gate signal and the second gate signal may be applied to different transistors included in the pixel circuit.

In an embodiment, the second transistor may further include a control electrode configured to receive the first gate signal, and the third transistor may further include a control electrode configured to receive the second gate signal.

In an embodiment, the gate driving circuit may include a normal output circuit configured to output a first gate signal at a first output node and control a voltage of a second output node in response to a previous first gate signal and a first clock signal and an inverted output circuit configured to output a third gate signal at a third output node in response to a second clock signal and the voltage of the second output node. The first gate signal and the third gate signal may be opposite in phase to each other, and the first gate signal and the third gate signal may be applied to different transistors included in the pixel circuit.

In an embodiment, the second transistor may further include a control electrode configured to receive the first gate signal, and the third transistor may further include a control electrode configured to receive the third gate signal.

In an embodiment of an electronic device according to the present inventive concept, the electronic device includes a display panel including a pixel circuit, a gate driving circuit configured to provide a gate signal to the pixel circuit, a data driving circuit configured to provide a data voltage to the pixel circuit, an emission driving circuit configured to provide an emission signal to the pixel circuit, a driving control circuit configured to control the gate driving circuit, the data driving circuit and the emission driving circuit based on an input control signal and an input image data, and a processor configured to output the input control signal and the input image data to the driving control circuit. The pixel circuit includes a light emitting element, a first transistor configured to provide a driving current to the light emitting element, a second transistor including a first electrode configured to receive a bias voltage and a second electrode connected to a first electrode of the first transistor and a third transistor including a first electrode connected to an anode electrode of the light emitting element and a second electrode configured to receive a first initialization voltage. One of the second transistor and the third transistor is a P-type transistor and an other one of the second transistor and the third transistor is an N-type transistor.

According to the gate driving circuit and the display device including the gate driving circuit, when the types (N-type or P-type) of the second transistor and the third transistor of the pixel circuit included in the display device are different and gate signals having phases opposite to phases of the gate signals are applied to the second transistor and the third transistor respectively, the driving voltage of the gate driving circuit may be decreased. When the driving voltage of the gate driving circuit is decreased, the power consumption of the display device may decrease. In addition, when two gate signals having opposite phases are output simultaneously from the gate driving circuit, the number of the gate driving circuits may be decreased and a dead space of the display device may be decreased.

### BRIEF DESCRIPTION OF THE DRAWINGS

The above and other features and advantages of the inventive concept will become more apparent by describing in detailed embodiments thereof with reference to the accompanying drawings.

FIG. 1 is a block diagram illustrating a display device according to an embodiment of the present inventive concept.

FIG. 2 is a circuit diagram illustrating a gate driving circuit of FIG. 1.

FIG. 3 is a timing diagram illustrating input signals and node signals of the gate driving circuit of FIG. 2.

FIG. 4 is a circuit diagram illustrating a pixel circuit of FIG. 1.

FIG. 5 is a circuit diagram illustrating a gate driving circuit of a display device according to an embodiment of the present inventive concept.

FIG. 6 is a timing diagram illustrating the gate driving circuit of FIG. 5.

FIG. 7 is a circuit diagram illustrating a pixel circuit of a display device according to an embodiment of the present inventive concept.

FIG. 8 is a circuit diagram illustrating a pixel circuit of a display device according to an embodiment of the present inventive concept.

FIG. 9 is a circuit diagram illustrating a pixel circuit of a display device according to an embodiment of the present inventive concept.

FIG. 10 is a circuit diagram illustrating a gate driving circuit of a display device according to an embodiment of the present inventive concept.

FIG. 11 is a circuit diagram illustrating a gate driving circuit of a display device according to an embodiment of the present inventive concept.

FIG. 12 is a circuit diagram illustrating a gate driving circuit of a display device according to an embodiment of the present inventive concept.

FIG. 13 is a circuit diagram illustrating a gate driving circuit of a display device according to an embodiment of the present inventive concept.

FIG. 14 is a circuit diagram illustrating a gate driving circuit of a display device according to an embodiment of the present inventive concept.

FIG. 15 is a circuit diagram illustrating a gate driving circuit of a display device according to an embodiment of the present inventive concept.

FIG. 16 is a circuit diagram illustrating a gate driving circuit of a display device according to an embodiment of the present inventive concept.

FIG. 17 is a circuit diagram illustrating a gate driving circuit of a display device according to an embodiment of the present inventive concept.

FIG. 18 is a circuit diagram illustrating a gate driving circuit of a display device according to an embodiment of the present inventive concept.

FIG. 19 is a circuit diagram illustrating a gate driving circuit of a display device according to an embodiment of the present inventive concept.

FIG. 20 is a block diagram illustrating an electronic device.

FIG. 21 is a diagram illustrating an embodiment in which the electronic device of FIG. 20 is implemented as a smart phone.

### DETAILED DESCRIPTION OF THE INVENTIVE CONCEPT

Hereinafter, the present inventive concept will be explained in detail with reference to the accompanying drawings.

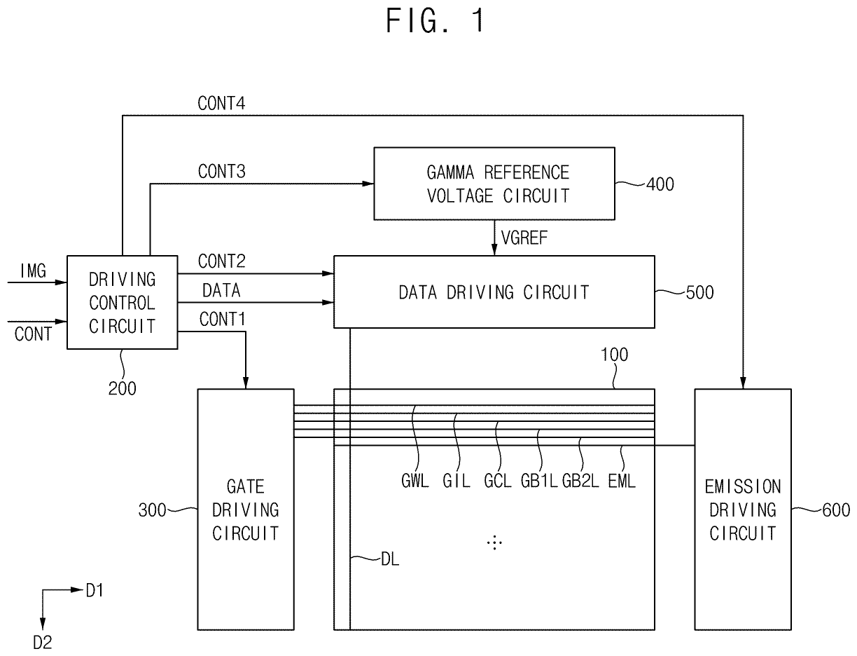

FIG. 1 is a block diagram illustrating a display device according to an embodiment of the present inventive concept.

Referring to FIG. 1, the display device includes a display panel 100 and display panel driving circuit. The display panel driving circuit includes a driving control circuit 200, a gate driving circuit 300, a gamma reference voltage generator 400, a data driving circuit 500 and an emission driving circuit 600.

The display panel 100 has a display region on which an image is displayed and a peripheral region adjacent to the display region.

The display panel 100 includes a plurality of gate lines GWL, GIL, GCL, GB1L and GB2L, a plurality of data lines DL, a plurality of emission lines EML and a plurality of pixels electrically connected to the gate lines GWL, GIL, GCL, GB1L and GB2L, the data lines DL and the emission lines EML. The gate lines GWL, GIL, GCL, GB1L and GB2L may extend in a first direction D1, the data lines DL may extend in a second direction D2 crossing the first direction D1 and the emission lines EML may extend in the first direction D1.

The driving control circuit 200 receives input image data IMG and an input control signal CONT from an external device. For example, the input image data IMG may include red image data, green image data and blue image data. The input image data IMG may include white image data. The input image data IMG may include magenta image data, cyan image data and yellow image data. The input control signal CONT may include a master clock signal and a data enable signal. The input control signal CONT may further include a vertical synchronizing signal and a horizontal synchronizing signal.

The driving control circuit 200 generates a first control signal CONT1, a second control signal CONT2, a third control signal CONT3, a fourth control signal CONT4 and a data signal DATA based on the input image data IMG and the input control signal CONT.

The driving control circuit 200 generates the first control signal CONT1 for controlling an operation of the gate driving circuit 300 based on the input control signal CONT, and outputs the first control signal CONT1 to the gate driving circuit 300. The first control signal CONT1 may include a vertical start signal and a gate clock signal.

The driving control circuit 200 generates the second control signal CONT2 for controlling an operation of the data driving circuit 500 based on the input control signal CONT, and outputs the second control signal CONT2 to the data driving circuit 500. The second control signal CONT2 may include a horizontal start signal and a load signal.

The driving control circuit 200 generates the data signal DATA based on the input image data IMG. The driving control circuit 200 outputs the data signal DATA to the data driving circuit 500.

The driving control circuit 200 generates the third control signal CONT3 for controlling an operation of the gamma reference voltage generator 400 based on the input control signal CONT, and outputs the third control signal CONT3 to the gamma reference voltage generator 400.

The driving control circuit 200 generates the fourth control signal CONT4 for controlling an operation of the emission driving circuit 600 based on the input control signal CONT, and outputs the fourth control signal CONT4 to the emission driving circuit 600.

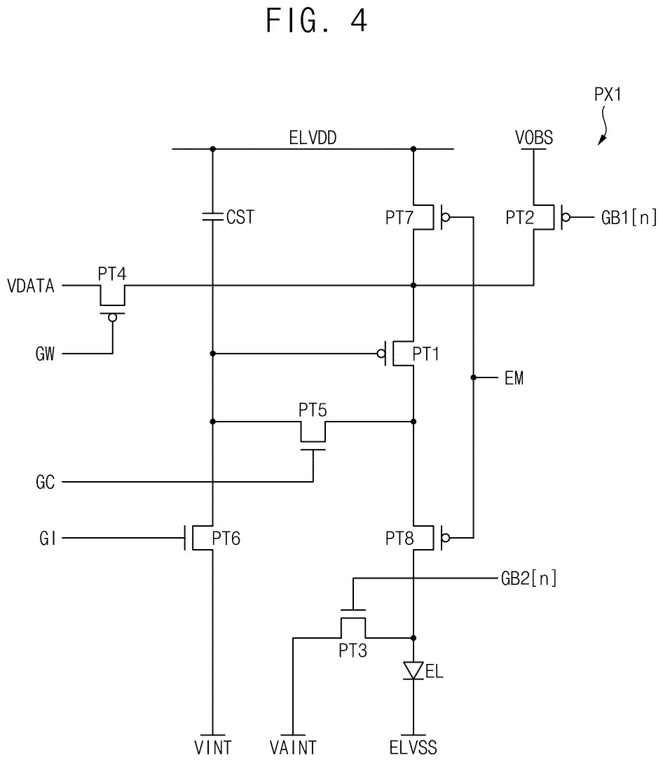

The gate driving circuit 300 generates gate signals driving the gate lines GWL, GIL, GCL and GBL in response to the first control signal CONT1 received from the driving control circuit 200. The gate driver 300 may output the gate signals to the gate lines GWL, GIL, GCL, GB1L and GB2L. The gate signals may include an initialization gate signal GI, a compensation gate signal GC, a writing gate signal GW, a first gate signal GB1 and a second gate signal GB2, see FIG. 4 for example.

In an embodiment of the present inventive concept, the gate driving circuit 300 may be integrated on the peripheral region of the display panel 100. In an embodiment of the present inventive concept, the gate driving circuit 300 may be mounted on the peripheral region of the display panel 100.

The gamma reference voltage generator 400 generates a gamma reference voltage VGREF in response to the third control signal CONT3 received from the driving control circuit 200. The gamma reference voltage generator 400 provides the gamma reference voltage VGREF to the data driving circuit 500. The gamma reference voltage VGREF has a value corresponding to a level of the data signal DATA.

In an embodiment, the gamma reference voltage generator 400 may be disposed in the driving control circuit 200, or in the data driving circuit 500.

The data driving circuit 500 receives the second control signal CONT2 and the data signal DATA from the driving control circuit 200, and receives the gamma reference voltages VGREF from the gamma reference voltage generator 400. The data driving circuit 500 converts the data signal DATA into data voltages having an analog type using the gamma reference voltages VGREF. The data driving circuit 500 outputs the data voltages to the data lines DL.

In an embodiment of the present inventive concept, the data driving circuit 500 may be integrated on the peripheral region of the display panel 100. In an embodiment of the present inventive concept, the data driving circuit 500 may be mounted on the peripheral region of the display panel 100.

The emission driving circuit 600 generates emission signals to drive the emission lines EML in response to the fourth control signal CONT4 received from the driving control circuit 200. The emission driving circuit 600 may output the emission signals to the emission lines EML.

In an embodiment of the present inventive concept, the emission driving circuit 600 may be integrated on the peripheral region of the display panel 100. In an embodiment of the present inventive concept, the emission driving circuit 600 may be mounted on the peripheral region of the display panel 100.

Although the gate driving circuit 300 is disposed at a first side of the display panel 100 and the emission driving circuit 600 is disposed at a second side of the display panel 100 opposite to the first side in FIG. 1 for convenience of explanation, the present inventive concept may not be limited thereto. For example, both of the gate driving circuit 300 and the emission driving circuit 600 may be disposed at the first side of the display panel 100. For example, the gate driving circuit 300 and the emission driving circuit 600 may be integrally formed.

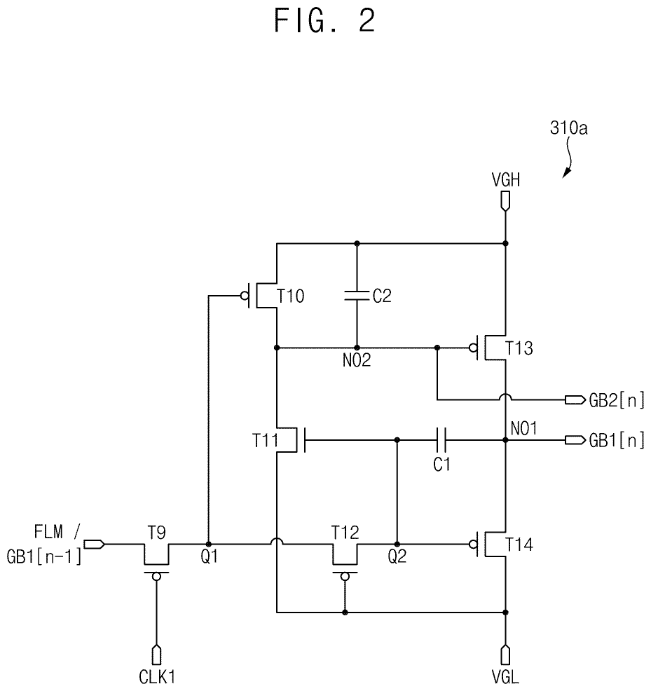

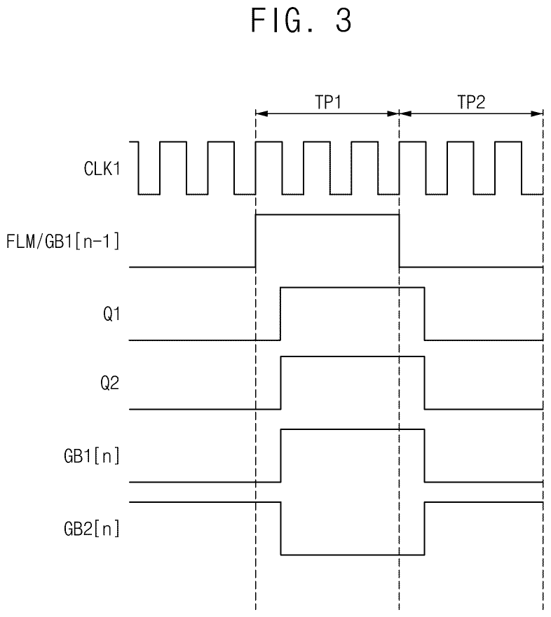

FIG. 2 is a circuit diagram illustrating the gate driving circuit 300 of FIG. 1. FIG. 3 is a timing diagram illustrating input signals and node signals of the gate driving circuit of FIG. 2.

Referring to FIGS. 1 to 3, the gate driving circuit 300 may include a normal output circuit 310a. The normal output circuit 310a may output the first gate signal GB1[n] at a first output node NO1 and a second gate signal GB2[n] at a second output node NO2 in response to a previous first gate signal GB1[n−1] and a first clock signal CLK1, wherein a phase of the first gate signal GB1[n] may be opposite to a phase of the second gate signal GB2[n], and wherein the first gate signal GB1[n] and the second gate signal GB2[n] may be applied to different transistors included in a pixel circuit.

In an embodiment of the present inventive concept, the normal output circuit 310a may include a ninth transistor T9, a tenth transistor T10, an eleventh transistor T11, a twelfth transistor T12, a thirteenth transistor T13, a fourteenth transistor T14, a first capacitor C1 and a second capacitor C2. The ninth transistor T9 may include a control electrode receiving the first clock signal CLK1, a first electrode receiving the previous first gate signal GB1[n−1] and a second electrode connected to a first node Q1. The tenth transistor T10 may include a control electrode connected to the first node Q1, a first electrode receiving a first gate power supply voltage VGH and a second electrode connected to the second output node NO2. The eleventh transistor T11 may include a first electrode connected to the second output node NO2 and a second electrode receiving a second gate power supply voltage VGL. The twelfth transistor T12 may include a control electrode receiving the second gate power supply voltage VGL, a first electrode connected to the first node Q1 and a second electrode connected to a second node Q2. The thirteenth transistor T13 may include a control electrode connected to the second output node NO2, a first electrode receiving the first gate power supply voltage VGH and a second electrode connected to the first output node NO1. The fourteenth transistor T14 may include a control electrode connected to the second node Q2, a first electrode connected to the first output node NO1 and a second electrode receiving the second gate power supply voltage VGL. The first capacitor C1 may include a first electrode connected to the second node Q2 and a second electrode connected to the first output node NO1. The second capacitor C2 may include a first electrode receiving the first gate power supply voltage VGH and a second electrode connected to the second output node NO2.

In an embodiment, when the normal output circuit 310a corresponds to a first pixel row, the previous first gate signal GB1[n−1] may be a vertical start signal FLM.

In an embodiment, a high voltage of the first clock signal CLK1, the previous first gate signal GB1[n−1], a voltage of the first node Q1, a voltage of the second node Q2, the first gate signal GB1[n] and the second gate signal GB2[n] may be the first gate power supply voltage VGH. In addition, a low voltage of the first clock signal CLK1, the previous first gate signal GB1[n−1], a voltage of the first node Q1, a voltage of the second node Q2, the first gate signal GB1[n] and the second gate signal GB2[n] may be the second gate power supply voltage VGL.

The first clock signal CLK1 may toggle between the high voltage VGH, sometimes called the first gate power supply voltage VGH, and the low voltage VGL, sometimes called the second gate power supply voltage VGL.

In a first period TP1, the previous first gate signal GB1[n−1] may increase to the high voltage VGH. After the previous first gate signal GB1[n−1] increases to the high voltage VGH, the ninth transistor T9 may turn on when the first clock signal CLK1 decreases to the low voltage VGL. The ninth transistor T9 may transfer the previous first gate signal GB1[n−1] having the high voltage VGH to the first node Q1. Therefore, the first node Q1 may have the high voltage VGH.

The twelfth transistor T12 may always be turned on by the second gate power supply voltage VGL. The twelfth transistor T12 may transfer the high voltage VGH of the first node Q1 to the second node Q2 and the voltage of the second node Q2 may be the high voltage VGH.

The eleventh transistor T11 may be turned on by the high voltage VGH of the second node Q2 and transfer the second gate power supply voltage VGL and a voltage of the second output node NO2 may be the low voltage VGL. The second output node NO2 may output the second gate signal GB2[n] having the low voltage VGL.

The fourteenth transistor T14 may be turned on by the high voltage VGH of the second node Q2 and the thirteenth transistor T13 may be turned on by the low voltage VGL of the second output node NO2. The thirteenth transistor T13 may transfer the first gate power supply voltage VGH to the first output node NO1 and the first output node NO1 may be output the first gate signal GB1[n] having the high voltage VGH.

In a second period TP2, the previous first gate signal GB1[n−1] may decrease from the high voltage VGH to the low voltage VGL. After the previous first gate signal GB1[n−1] decreases to the low voltage VGL, when the first clock signal CLK1 decreases to the low voltage VGL, the ninth transistor T9 may be turned on and transfer the previous first gate signal GB1[n−1] having the low voltage VGL to the first node Q1. Therefore, a voltage of the first node Q1 may be the low voltage VGL.

The twelfth transistor T12 may always be turned on by the second gate power supply voltage VGL. The twelfth transistor T12 may transfer the low voltage VGL of the first node Q1 to the second node Q2, and the second node Q2 may have the low voltage VGL. The eleventh transistor T11 may be turned on by the low voltage VGL of the second node Q2.

The tenth transistor T10 may be turned on by the low voltage VGL of the first node Q1. The tenth transistor T10 may transfer the first gate power supply voltage VGH to the second output node NO2, and the output node NO2 may have the high voltage VGH. Therefore, the second output node NO2 may output the second gate signal GB2[n] having the high voltage VGH.

The thirteenth transistor T13 may be turned off by the high voltage VGH of the second output node NO2, and the fourteenth transistor T14 may be turned on by the low voltage VGL of the second node Q2. The fourteenth transistor T14 may transfer the second gate power supply voltage VGL to the first output node NO1. The first output node NO1 may have the low voltage VGL and output the first gate signal GB1[n] having the low voltage VGL.

When the voltage of the first output node NO1 decreases from the high voltage VGH to the low voltage VGL, the second node Q2 may be bootstrapped by the first capacitor C1.

When the voltage of the second node Q2 is bootstrapped, a channel current of the twelfth transistor T12 may be zero. In other words, the twelfth transistor T12 may be turned off while the voltage of the second node Q2 is bootstrapped and disconnect the electrical connection between the first node Q1 and the control electrode of the fourteenth transistor T14. In addition, the twelfth transistor T12 may be turned on while the voltage of the second node Q2 is not bootstrapped and electrically connect the first node Q1 and the control electrode of the fourteenth transistor T14.

The second capacitor C2 may stabilize the voltage of the second output node NO2.

When the first gate signal GB1[n] and the second gate signal GB2[n], which have opposite phases, are output simultaneously from the gate driving circuit 300 to the pixel circuit, a separate gate driving circuit is not required. Accordingly, the number of gate driving circuits in the display device may be reduced, and a dead space of the display device may be reduced.

FIG. 4 is a circuit diagram illustrating a pixel circuit of FIG. 1.

Referring to FIGS. 1 to 4, the pixel circuit PX1 may include a light emitting element EL, a first pixel transistor PT1, a second pixel transistor PT2 and a third pixel transistor PT3.

The first pixel transistor PT1 may apply driving current to the light emitting element EL.

The second pixel transistor PT2 may include a first electrode receiving a bias voltage VOBS and a second electrode connecting to a first electrode of the first pixel transistor PT1.

The third pixel transistor PT3 may include a first electrode connected to an anode electrode of the light emitting element EL and a second electrode receiving a first initialization voltage VAINT.

At least one of the second pixel transistor PT2 and the third pixel transistor PT3 may be an N-type transistor, and the other of the second pixel transistor PT2 and the third pixel transistor PT3 may be a P-type transistor. For example, the second pixel transistor PT2 may be the P-type transistor, and the third pixel transistor PT3 may be the N-type transistor.

The pixel circuit PX1 may include a fourth pixel transistor PT4, a fifth pixel transistor PT5, a sixth pixel transistor PT6, a seventh pixel transistor PT7, a eighth pixel transistor PT8 and storage capacitor CST.

The fourth pixel transistor PT4 may include a control electrode receiving a writing gate signal GW, a first electrode receiving a data voltage VDATA, and a second electrode connected to the first electrode of the first pixel transistor PT1.

The fifth pixel transistor PT5 may include a control electrode receiving a compensation gate signal GC, a first electrode connected to the control electrode of the first pixel transistor PT1, and a second electrode connected to a second electrode of the first pixel transistor PT1.

The sixth pixel transistor PT6 may include a control electrode receiving an initialization gate signal GI, a second electrode connected to the control electrode of the first pixel transistor PT1, and a second electrode receiving a second initialization voltage VINT.

The seventh pixel transistor PT7 may include a control electrode receiving a emission signal EM, a first electrode receiving a first power supply voltage ELVDD, and a second electrode connected to the first electrode of the first pixel transistor PT1.

The eighth pixel transistor PT8 may include a control electrode receiving the emission signal EM, a first electrode connected to the second electrode of the first pixel transistor PT1, and a second electrode connected to the first electrode of the third pixel transistor PT3.

The storage capacitor CST may include a first electrode receiving the first power supply voltage ELVDD, and a second electrode connected to the control electrode of the first pixel transistor PT1.

A cathode electrode of the light emitting element EL may receive a second power supply voltage ELVSS.

The normal output circuit 310a of the gate driving circuit 300 in the FIG. 2 may output the first gate signal GB1[n] at the first output node NO1 and the second gate signal GB2[n] at a second output node NO2 in response to the previous first gate signal GB1[n−1] and the first clock signal CLK1, wherein the phase of the first gate signal GB1[n] may be opposite to the phase of the second gate signal GB2[n].

The second pixel transistor PT2 of the pixel circuit PX1 may further include a control electrode receiving the first gate signal GB1[n] of the normal output circuit 310a.

The third pixel transistor PT3 of the pixel circuit PX1 may further include a control electrode receiving the second gate signal GB2[n] of the normal output circuit 310a.

The bias voltage VOBS applied to the first electrode of the second pixel transistor PT2 of the pixel circuit PX1 may have a positive voltage. The first initialization voltage VAINT applied to the second electrode of the third pixel transistor PT3 of the pixel circuit PX1 may have a negative voltage. When the first gate signal GB1[n] and the second gate signal GB2[n] are output from the normal output circuit 310a, a high voltage of the first gate signal GB1[n] and a low voltage of the second gate signal GB2[n] may have a same magnitude.

When the second pixel transistor PT2 is the P-type transistor, the second pixel transistor PT2 may be turned on when the first gate signal GB1[n] have the low voltage VGL. When the third pixel transistor PT3 is the P-type transistor, the third pixel transistor PT3 may be turned on when the second gate signal GB2[n] have the low voltage VGL.

When the second pixel transistor PT2 and the third pixel transistor PT3 are P-type transistor, the low voltage VGL may be a higher voltage for the second pixel transistor PT2 than for the third pixel transistor PT3. That is, the second pixel transistor PT2 may have a smaller magnitude of the low voltage VGL than the third pixel transistor PT3.

When the second pixel transistor PT2 is the N-type transistor, the second pixel transistor PT2 may be turned on when the first gate signal GB1[n] have the high voltage VGH.

When the third pixel transistor PT3 is the N-type transistor, the third pixel transistor PT3 may be turned on when the second gate signal GB2[n] have the high voltage VGH.

When the second pixel transistor PT2 and the third pixel transistor PT3 are N-type transistor, the high voltage VGH may be a lower voltage for the third pixel transistor PT3 than for the second pixel transistor PT2. That is, the third pixel transistor PT3 may have a smaller magnitude of the high voltage VGH than the second pixel transistor PT2.

When the second pixel transistor PT2 is the P-type transistor and the third pixel transistor PT3 is the N-type transistor, the magnitude of the high voltage VGH may be smaller than when the second pixel transistor PT2 and the third pixel transistor PT3 are N-type transistors. That is, a magnitude of a driving voltage of the normal output circuit 310a outputting the first gate signal GB1[n] and the second gate signal GB2[n] may be reduced. Power consumption of the display device may be reduced.

When the second pixel transistor PT2 is the P-type transistor and the third pixel transistor PT3 is the N-type transistor, the magnitude of the low voltage VGL may be smaller than when the second pixel transistor PT2 and the third pixel transistor PT3 are P-type transistors. That is, the magnitude of the driving voltage of the normal output circuit 310a outputting the first gate signal GB1[n] and the second gate signal GB2[n] may be reduced. Power consumption of the display device may be reduced.

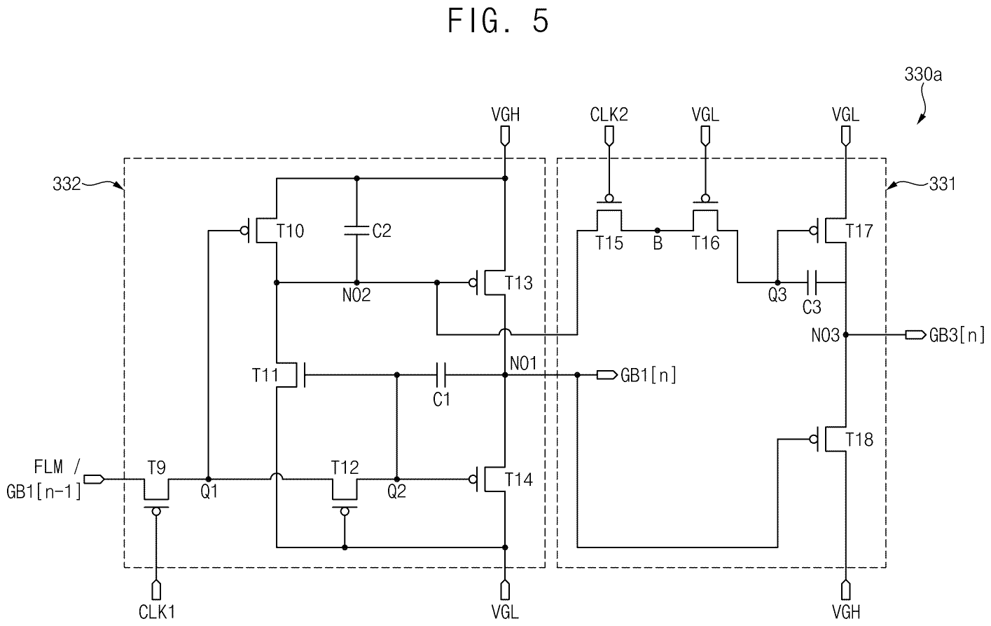

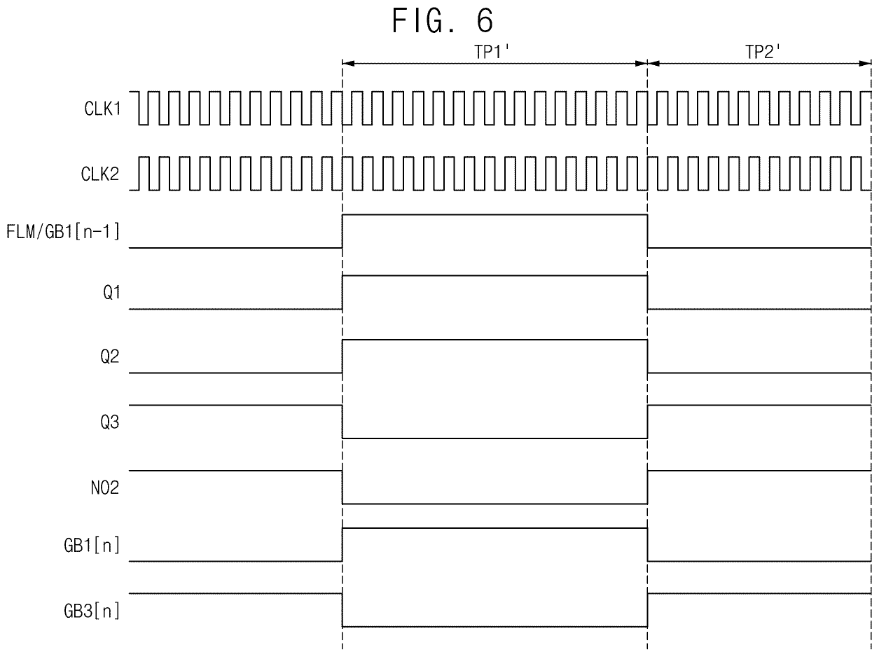

FIG. 5 is a circuit diagram illustrating a gate driving circuit 330a of the display device according to an embodiment of the present inventive concept. FIG. 6 is a timing diagram illustrating the gate driving circuit of FIG. 5.

The gate driving circuit 330a according to the present embodiment is substantially the same as the gate driving circuit 310a of the previous embodiment explained referring to FIG. 2 except that it further includes an inverted output circuit 331, and a connection relationship and driving method of a normal output circuit 332 and the inverted output circuit 331 are different. Thus, the same reference numerals will be used to refer to the same or like parts as those described in the previous embodiment of FIG. 2 and any repetitive explanation concerning the above elements will be omitted.

Referring to FIGS. 1 and 4 to 6, the gate driving circuit 330a may include the normal output circuit 332 and the inverted output circuit 331.

The normal output circuit 332 may output the first gate signal GB1[n] at the first output node NO1 in response to the previous first gate signal GB1[n−1] and the first clock signal CLK1, and control the voltage of the second output node NO2.

The inverted output circuit 331 may output a third gate signal GB3[n] at the gate output node NO3 in response to the second clock signal CLK2 and the voltage of the second output node NO2.

A phase of the first gate signal GB1[n] may be opposite to a phase of the third gate signal GB3[n], and the first gate signal GB1[n] and the third gate signal GB3[n] may be applied to different transistors included in the pixel circuit PX1.

In an embodiment of the present inventive concept, the inverted output circuit 331 may include a fifteenth transistor T15, a sixteenth transistor T16, a seventeenth transistor T17, a eighteenth transistor T18 and a third capacitor C3.

The fifteenth transistor T15 may include a control electrode receiving the second clock signal CLK2, a first electrode connected to the second output node NO2 and a second electrode connected to a buffer node B.

The sixteenth transistor T16 may include a control electrode receiving the second gate power supply voltage VGL, a first electrode connected to the buffer node B and a second electrode connected to a third node Q3.

The seventeenth transistor T17 may have a control electrode connected to the third node Q3, a first electrode receiving the second gate power supply voltage VGL and a second electrode connected to the third output node NO3.

The eighteenth transistor T18 may include a control electrode connected to the first output node NO1, a first electrode connected to the third output node NO3 and a second electrode receiving the gate power supply voltage VGH.

The third capacitor C3 may include a first electrode connected to the third node Q3 and a second electrode connected to the third output node NO3.

In a first period TP1′, the previous first gate signal GB1[n−1] may increase from the low voltage VGL to the high voltage VGH. At this time, the first node Q1 and the second node Q2 may have the high voltage VGH. In addition, the second output node NO2 may have the low voltage VGL, and the first output node NO1 may output the first gate signal GB1[n] having the high voltage VGH.

When the second clock signal CLK2 decreases from the high voltage VGH to the low voltage, the fifteenth transistor T15 may be turned on. The fifteenth transistor T15 may transfer the low voltage VGL of the second output node NO2 to the buffer node B. The buffer node B may have the low voltage VGL.

The sixteenth transistor T16 may always be turned by the second gate power supply voltage VGL. The sixteenth transistor T16 may transfer the low voltage VGL of the buffer node B to the third node Q3.

The eighteenth transistor T18 may be turned off by the high voltage VGH of the first output node NO1. The seventeenth transistor T17 may be turned on by the low voltage VGL of the third node Q3. The seventeenth transistor T17 may transfer the second gate power supply voltage VGL to the third output node NO3. The third output node NO3 may have the low voltage VGL. Accordingly, the third output node NO3 may output the third gate signal GB3[n] having the low voltage VGL.

In a second period TP2′, the previous first gate signal GB1[n−1] may decrease from the high voltage VGH to the low voltage VGL. At this time, the first node Q1 and the second node Q2 may have the low voltage VGL. In addition, the second output node NO2 may have the high voltage VGH, and the first output node NO1 may output the first gate signal GB1[n] having the low voltage VGL.

When the second clock signal CLK2 decreases from the high voltage VGH to the low voltage, the fifteenth transistor T15 may be turned on. The fifteenth transistor T15 may transfer the high voltage VGH of the second output node NO2 to the buffer node B. The buffer node B may have the high voltage VGH.

The sixteenth transistor T16 may always be turned by the second gate power supply voltage VGL. The sixteenth transistor T16 may transfer the high voltage VGH of the buffer node B to the third node Q3.

The eighteenth transistor T18 may be turned on by the low voltage VGL of the first output node NO1. The seventeenth transistor T17 may be turned off by the high voltage VGH of the third node Q3. The eighteenth transistor T18 may transfer the first gate power supply voltage VGH to the third output node NO3. The third output node NO3 may have the high voltage VGH. Accordingly, the third output node NO3 may output the third gate signal GB3[n] having the high voltage VGH.

When the voltage of the third output node NO3 decreases from the high voltage VGH to the low voltage VGL, the third node Q3 may be bootstrapped by the third capacitor C3.

The channel current of the sixteenth transistor T16 may be zeroed when the voltage of the third node Q3 is bootstrapped. That is, the sixteenth transistor T16 may be turned off while the voltage of the third node Q3 is bootstrapped. The electrical connection may be cut off between the buffer node B and the control electrode of the seventeenth transistor T17. In additional, the sixteenth transistor T16 may be turned on while the voltage of the third node Q3 is not bootstrapped. The buffer node B and the control electrode of the seventeenth transistor T17 may be electrically connected.

When the first gate signal GB1[n] and the third gate signal GB3[n], which have opposite phases, are output simultaneously from the gate driving circuit 330a to the pixel circuit PX1, a separate gate driving circuit is not required. Accordingly, the number of gate driving circuits in the display device may be reduced, and a dead space of the display device may be reduced.

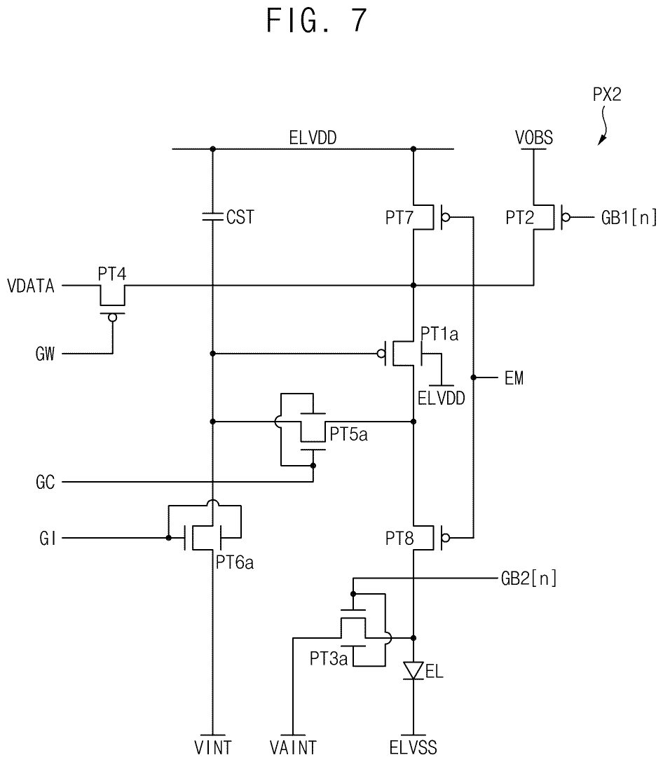

FIG. 7 is a circuit diagram illustrating a pixel circuit PX2 of a display device according to an embodiment of the present inventive concept.

The pixel circuit PX2 according to the present embodiment is substantially the same as the pixel circuit PX1 of the previous embodiment explained referring to FIG. 4 except that a first pixel transistor PT1a, a third pixel transistor PT3a, a fifth pixel transistor PT5a and a sixth pixel transistor PT6a further include a second control electrode. Thus, the same reference numerals will be used to refer to the same or like parts as those described in the previous embodiment of FIG. 4 and any repetitive explanation concerning the above elements will be omitted.

Referring to FIGS. 1 to 4 and 7, the third pixel transistor PT3a may further include the second control electrode connected to the control electrode of the third pixel transistor PT3a. The fifth pixel transistor PT5a may further include the second control electrode connected to the control electrode of the fifth pixel transistor PT5a. The sixth pixel transistor PT6a may further include the second control electrode connected to the control electrode of the sixth pixel transistor PT6a.

When the second control electrode is connected to the control electrode, the threshold voltage shift caused by transistor stress may be prevented, and the leakage current of the transistor may be reduced. Accordingly, the stability and reliability of the pixel circuit PX2 may be increased.

The first pixel transistor PT1a may further include the second control electrode receiving the first power supply voltage ELVDD.

When the first power supply voltage ELVDD applied to the second control electrode of the first pixel transistor PT1a, the threshold voltage shift of the first pixel transistor PT1a caused by transistor stress may be prevented, and the leakage current of the first pixel transistor PT1a may be reduced. Accordingly, the stability and reliability of the pixel circuit PX2 may be increased.

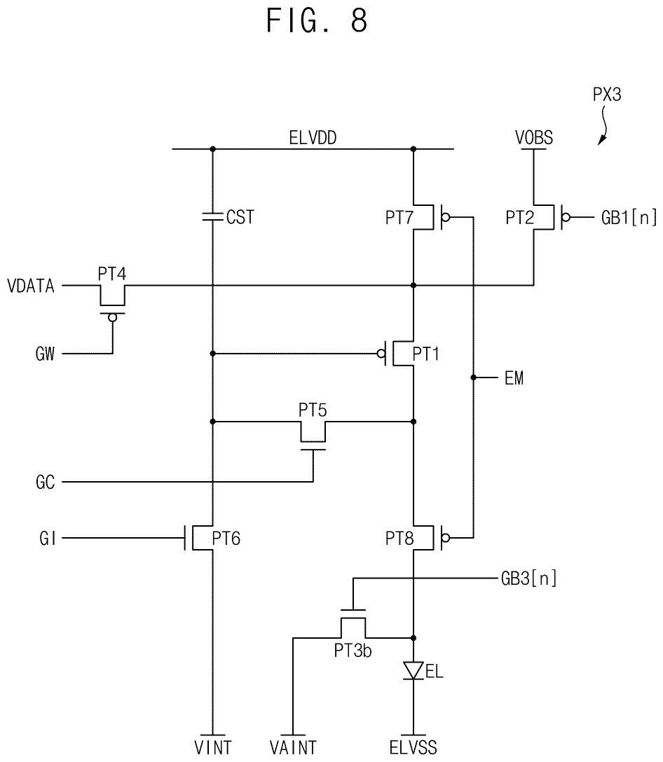

FIG. 8 is a circuit diagram illustrating a pixel circuit PX3 of a display device according to an embodiment of the present inventive concept.

The pixel circuit PX3 according to the present embodiment is substantially the same as the pixel circuit PX1 of the previous embodiment explained referring to FIG. 4 except that the third gate signal GB3[n] is applied to the control electrode of the third pixel transistor PT3b. Thus, the same reference numerals will be used to refer to the same or like parts as those described in the previous embodiment of FIG. 4 and any repetitive explanation concerning the above elements will be omitted.

Referring to FIGS. 1, 4 to 6 and 8, the normal output circuit 332 of the gate driving circuit 330a may output the first gate signal GB1[n] at the first output node NO1 in response to the previous first gate signal GB1[n−1] and the first clock signal CLK1. The inverted output circuit 331 may output a third gate signal GB3[n] at the second output node NO3 in response to the second clock signal CLK2 and the voltage of the second output node NO2. The phase of the first gate signal GB1[n] may be opposite to the phase of the third gate signal GB3[n].

The third pixel transistor PT3b of the pixel circuit PX3 may further include a control electrode receiving the third gate signal GB3[n] output from the third output node NO3 of the inverted output circuit 331.

The magnitude of the driving voltage of the gate driving circuit 310a outputting the first gate signal GB1[n] and the third gate signal GB3[n] may be decreased. Accordingly, the power consumption of the display device may be decreased.

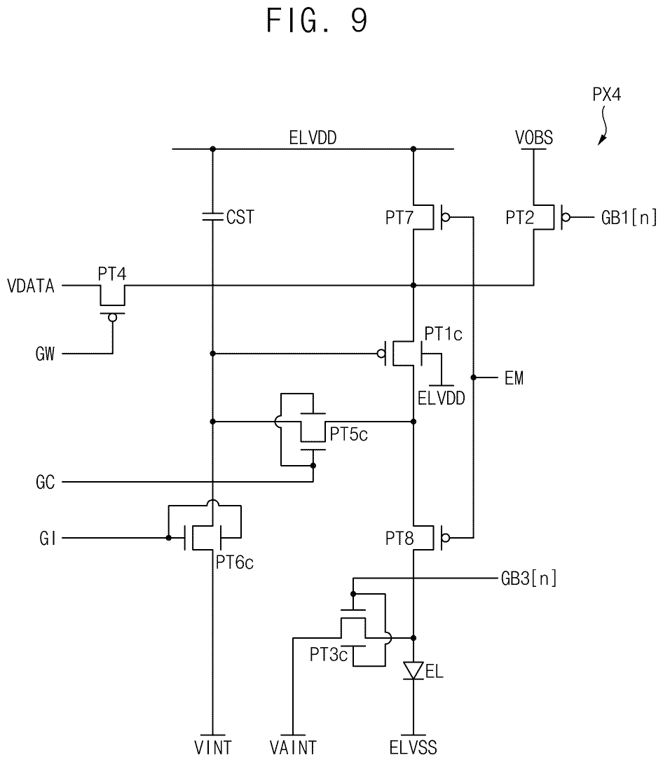

FIG. 9 is a circuit diagram illustrating a pixel circuit PX4 of a display device according to an embodiment of the present inventive concept.

The pixel circuit PX4 according to the present embodiment is substantially the same as the pixel circuit PX3 of the previous embodiment explained referring to FIG. 8 except that a first pixel transistor PT1c, a third pixel transistor PT3c, a fifth pixel transistor PT5c and a sixth pixel transistor PT6c further include a second control electrode. Thus, the same reference numerals will be used to refer to the same or like parts as those described in the previous embodiment of FIG. 8 and any repetitive explanation concerning the above elements will be omitted.

Referring to FIGS. 1, 4 to 6, 8 and 9, the third pixel transistor PT3c may further include the second control electrode connected to the control electrode of the third pixel transistor PT3c. The fifth pixel transistor PT5c may further include the second control electrode connected to the control electrode of the fifth pixel transistor PT5c. The sixth pixel transistor PT6c may further include the second control electrode connected to the control electrode of the sixth pixel transistor PT6c.

When the second control electrode is connected to the control electrode, the threshold voltage shift caused by transistor stress may be prevented, and the leakage current of the transistor may be reduced. Accordingly, the stability and reliability of the pixel circuit PX4 may be increased.

The first pixel transistor PT1c may further include the second control electrode receiving the first power supply voltage ELVDD.

When the first power supply voltage ELVDD applied to the second control electrode of the first pixel transistor PT1c, the threshold voltage shift of the first pixel transistor PT1c caused by transistor stress may be prevented, and the leakage current of the first pixel transistor PT1c may be reduced. Accordingly, the stability and reliability of the pixel circuit PX4 may be increased.

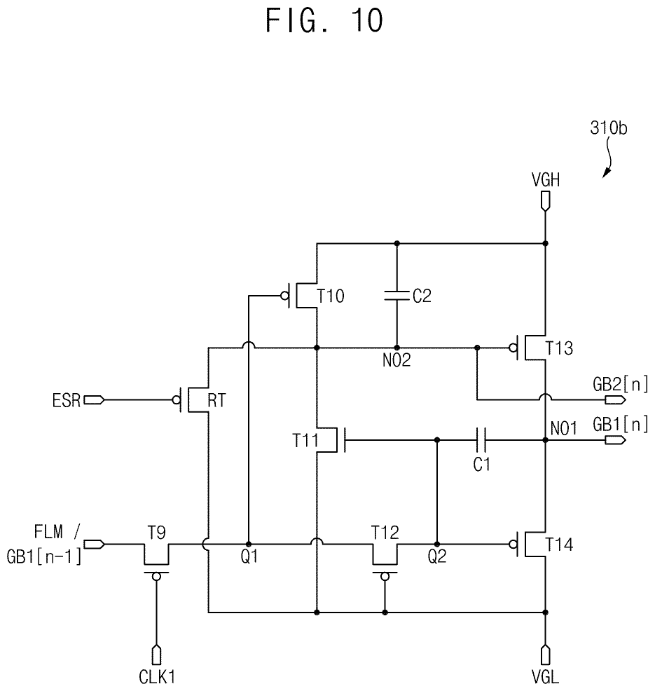

FIG. 10 is a circuit diagram illustrating a gate driving circuit 310b of a display device according to an embodiment of the present inventive concept.

The gate driving circuit 310b according to the present embodiment is substantially the same as the gate driving circuit 310a of the previous embodiment explained referring to FIG. 2 except that the gate driving circuit 310b further include a reset transistor RT. Thus, the same reference numerals will be used to refer to the same or like parts as those described in the previous embodiment of FIG. 2 and any repetitive explanation concerning the above elements will be omitted.

Referring to FIGS. 1 to 3 and 10, the gate driving circuit 310b may further include the reset transistor RT.

The reset transistor RT may include a control electrode receiving a reset signal ESR, a first electrode connected to the second output node NO2 and second electrode receiving the second gate power supply voltage VGL.

When the reset signal ESR has the low voltage VGL, the reset transistor RT may be turned on. The reset transistor RT may transfer the second gate power supply voltage VGL to the second output node NO2. When the second output node NO2 has the low voltage VGL, the thirteenth transistor T13 may be turned on. The thirteenth transistor T13 may transfer the first gate power supply voltage VGH to the first output node NO1. The first output node NO1 may have the high voltage VGH.

When the display device is first driven, the reset transistor RT may reset the voltage of the output node NO2 to the low voltage VGL. Accordingly, the stability and reliability of the gate driving circuit 310b may be increased.

When the first gate signal GB1[n] and the second gate signal GB2[n], which have opposite phases, are output simultaneously from the gate driving circuit 310b to the pixel circuit, a separate gate driving circuit is not required. Accordingly, the number of gate driving circuits in the display device may be reduced, and the dead space of the display device may be reduced.

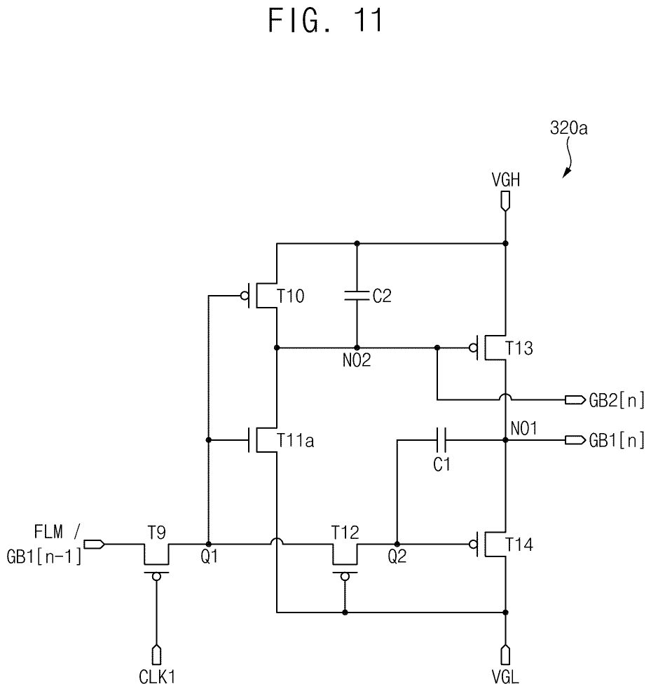

FIG. 11 is a circuit diagram illustrating a gate driving circuit 320a of a display device according to an embodiment of the present inventive concept.

The gate driving circuit 320a according to the present embodiment is substantially the same as the gate driving circuit 310a of the previous embodiment explained referring to FIG. 2 except that a control electrode of the eleventh transistor T11a is connected to the first node Q1. Thus, the same reference numerals will be used to refer to the same or like parts as those described in the previous embodiment of FIG. 2 and any repetitive explanation concerning the above elements will be omitted.

Referring to FIGS. 1 to 3 and 11, the eleventh transistor T11a may include the control electrode connected to the first node Q1, the first electrode connected to the second output node NO2 and the second electrode receiving the second gate power supply voltage VGL.

When the first gate signal GB1[n] and the second gate signal GB2[n], which have opposite phases, are output simultaneously from the gate driving circuit 320a to the pixel circuit, a separate gate driving circuit is not required. Accordingly, the number of gate driving circuits in the display device may be reduced, and the dead space of the display device may be reduced.

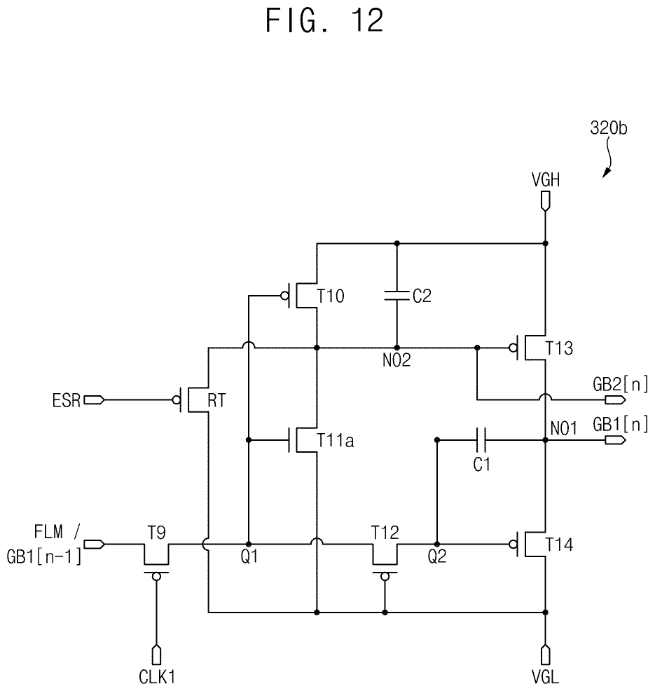

FIG. 12 is a circuit diagram illustrating a gate driving circuit 320b of a display device according to an embodiment of the present inventive concept.

The gate driving circuit 320b according to the present embodiment is substantially the same as the gate driving circuit 320a of the previous embodiment explained referring to FIG. 11 except that the gate driving circuit 320b further include a reset transistor RT. Thus, the same reference numerals will be used to refer to the same or like parts as those described in the previous embodiment of FIG. 11 and any repetitive explanation concerning the above elements will be omitted.

Referring to FIGS. 1 to 3, 11 and 12, the gate driving circuit 320b may further include the reset transistor RT.

The reset transistor RT may include a control electrode receiving a reset signal ESR, a first electrode connected to the second output node NO2 and second electrode receiving the second gate power supply voltage VGL.

When the reset signal ESR has the low voltage VGL, the reset transistor RT may be turned on. The reset transistor RT may transfer the second gate power supply voltage VGL to the second output node NO2. When the second output node NO2 has the low voltage VGL, the thirteenth transistor T13 may be turned on. The thirteenth transistor T13 may transfer the first gate power supply voltage VGH to the first output node NO1. The first output node NO1 may have the high voltage VGH.

When the display device is first driven, the reset transistor RT may reset the voltage of the output node NO2 to the low voltage VGL. Accordingly, the stability and reliability of the gate driving circuit 320b may be increased.

When the first gate signal GB1[n] and the second gate signal GB2[n], which have opposite phases, are output simultaneously from the gate driving circuit 320b to the pixel circuit, a separate gate driving circuit is not required. Accordingly, the number of gate driving circuits in the display device may be reduced, and the dead space of the display device may be reduced.

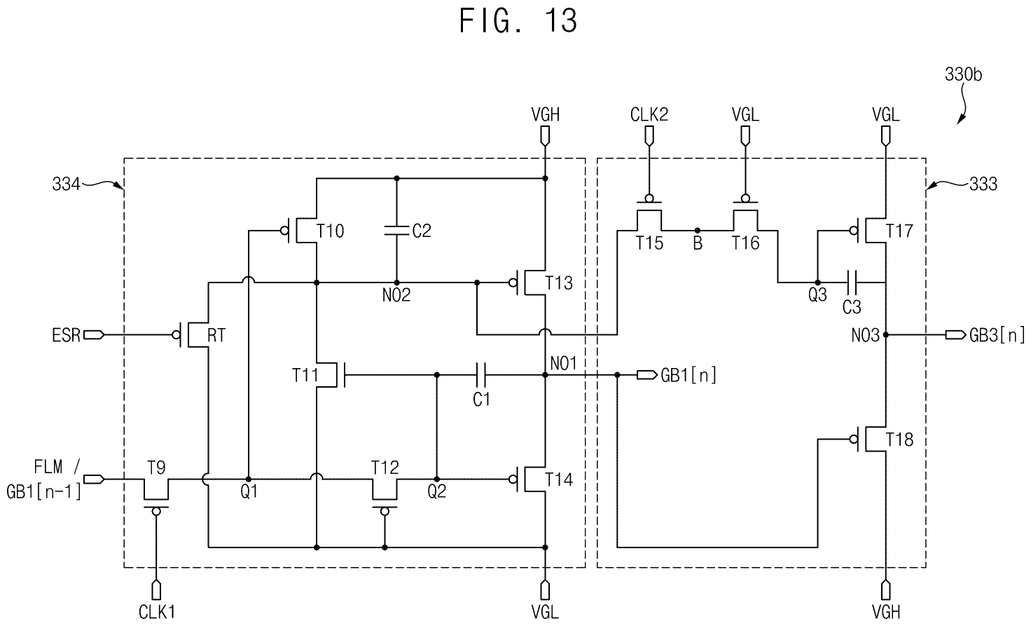

FIG. 13 is a circuit diagram illustrating a gate driving circuit 330b of a display device according to an embodiment of the present inventive concept.

The gate driving circuit 330b according to the present embodiment is substantially the same as the gate driving circuit 330a of the previous embodiment explained referring to FIG. 5 except that the gate driving circuit 330b further include a reset transistor RT. Thus, the same reference numerals will be used to refer to the same or like parts as those described in the previous embodiment of FIG. 5 and any repetitive explanation concerning the above elements will be omitted.

Referring to FIGS. 1, 4 to 6 and 13, the gate driving circuit 330b may further include the reset transistor RT.

The reset transistor RT may include a control electrode receiving a reset signal ESR, a first electrode connected to the second output node NO2 and second electrode receiving the second gate power supply voltage VGL.

When the reset signal ESR has the low voltage VGL, the reset transistor RT may be turned on. The reset transistor RT may transfer the second gate power supply voltage VGL to the second output node NO2. When the second output node NO2 has the low voltage VGL, the thirteenth transistor T13 may be turned on. The thirteenth transistor T13 may transfer the first gate power supply voltage VGH to the first output node NO1. The first output node NO1 may have the high voltage VGH.

When the display device is first driven, the reset transistor RT may reset the voltage of the output node NO2 to the low voltage VGL. Accordingly, the stability and reliability of the gate driving circuit 330b may be increased.

When the first gate signal GB1[n] and the third gate signal GB3[n], which have opposite phases, are output simultaneously from the gate driving circuit 330b to the pixel circuit, a separate gate driving circuit is not required. Accordingly, the number of gate driving circuits in the display device may be reduced, and the dead space of the display device may be reduced.

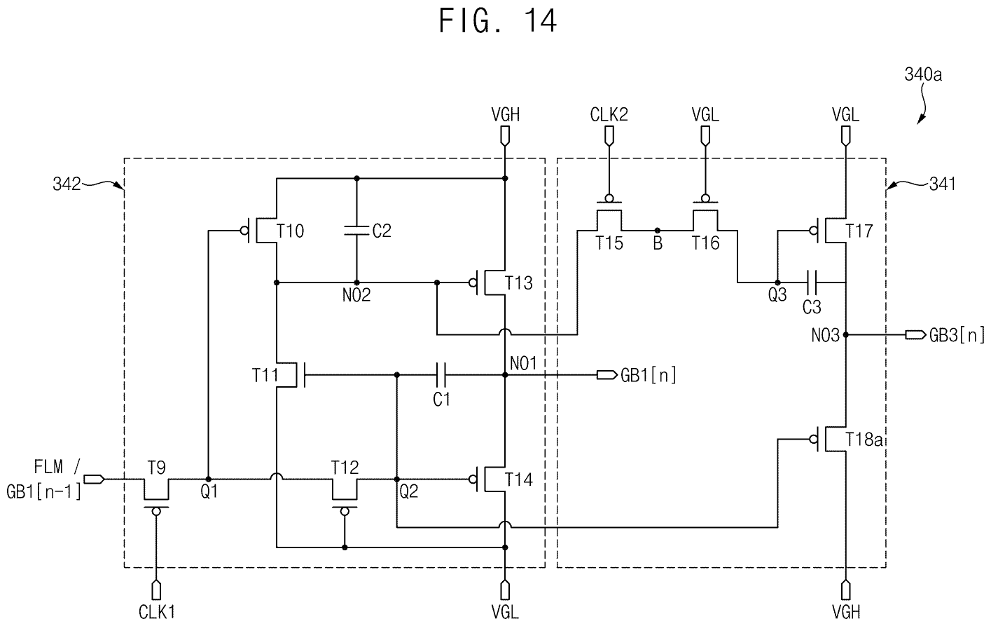

FIG. 14 is a circuit diagram illustrating a gate driving circuit 340a of a display device according to an embodiment of the present inventive concept.

The gate driving circuit 340a according to the present embodiment is substantially the same as the gate driving circuit 330a of the previous embodiment explained referring to FIG. 5 except that a control electrode of an eighteenth transistor T18a is connected to the second node Q2. Thus, the same reference numerals will be used to refer to the same or like parts as those described in the previous embodiment of FIG. 5 and any repetitive explanation concerning the above elements will be omitted.

Referring to FIGS. 1, 4 to 6 and 14, the eighteenth transistor T18a of the gate driving circuit 340a may include the control electrode connected to the second node Q2, the first electrode connected to the third output node NO3 and the second electrode receiving the first gate power supply voltage VGH.

When the first gate signal GB1[n] and the third gate signal GB3[n], which have opposite phases, are output simultaneously from the gate driving circuit 340a to the pixel circuit, a separate gate driving circuit is not required. Accordingly, the number of gate driving circuits in the display device may be reduced, and the dead space of the display device may be reduced.

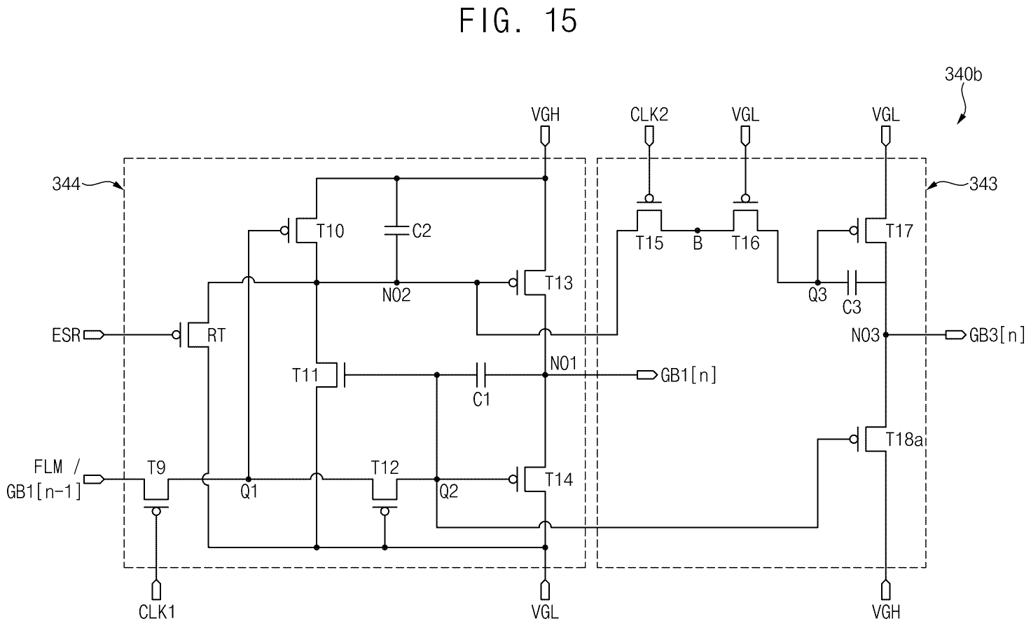

FIG. 15 is a circuit diagram illustrating a gate driving circuit 340b of a display device according to an embodiment of the present inventive concept.

The gate driving circuit 340b according to the present embodiment is substantially the same as the gate driving circuit 340a of the previous embodiment explained referring to FIG. 14 except that the gate driving circuit 340b further include a reset transistor RT. Thus, the same reference numerals will be used to refer to the same or like parts as those described in the previous embodiment of FIG. 14 and any repetitive explanation concerning the above elements will be omitted.

Referring to FIGS. 1, 4 to 6, 14 and 15, the gate driving circuit 340b may further include the reset transistor RT.

The reset transistor RT may include a control electrode receiving a reset signal ESR, a first electrode connected to the second output node NO2 and second electrode receiving the second gate power supply voltage VGL.

When the reset signal ESR has the low voltage VGL, the reset transistor RT may be turned on. The reset transistor RT may transfer the second gate power supply voltage VGL to the second output node NO2. When the second output node NO2 has the low voltage VGL, the thirteenth transistor T13 may be turned on. The thirteenth transistor T13 may transfer the first gate power supply voltage VGH to the first output node NO1. The first output node NO1 may have the high voltage VGH.

When the display device is first driven, the reset transistor RT may reset the voltage of the output node NO2 to the low voltage VGL. Accordingly, the stability and reliability of the gate driving circuit 340b may be increased.

When the first gate signal GB1[n] and the third gate signal GB3[n], which have opposite phases, are output simultaneously from the gate driving circuit 340b to the pixel circuit, a separate gate driving circuit is not required. Accordingly, the number of gate driving circuits in the display device may be reduced, and the dead space of the display device may be reduced.

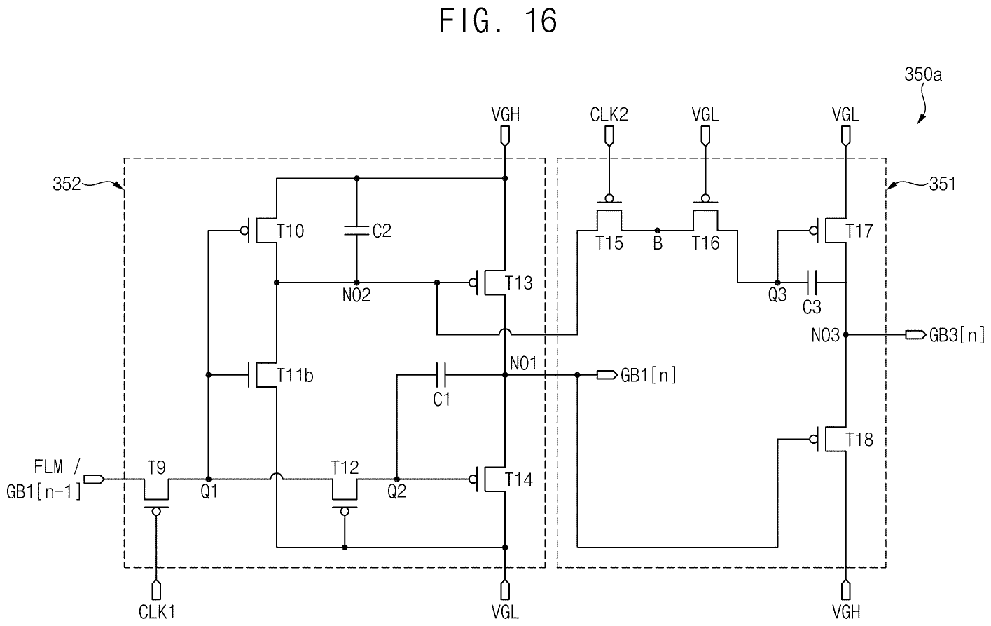

FIG. 16 is a circuit diagram illustrating a gate driving circuit 350a of a display device according to an embodiment of the present inventive concept.

The gate driving circuit 350a according to the present embodiment is substantially the same as the gate driving circuit 330a of the previous embodiment explained referring to FIG. 5 except that a control electrode of the eleventh transistor T11b is connected to the first node Q1. Thus, the same reference numerals will be used to refer to the same or like parts as those described in the previous embodiment of FIG. 5 and any repetitive explanation concerning the above elements will be omitted.

Referring to FIGS. 1, 4 to 6 and 16, the eleventh transistor T11b may include the control electrode connected to the first node Q1, the first electrode connected to the second output node NO2 and the second electrode receiving the second gate power supply voltage VGL.

When the first gate signal GB1[n] and the third gate signal GB3[n], which have opposite phases, are output simultaneously from the gate driving circuit 350a to the pixel circuit, a separate gate driving circuit is not required. Accordingly, the number of gate driving circuits in the display device may be reduced, and the dead space of the display device may be reduced.

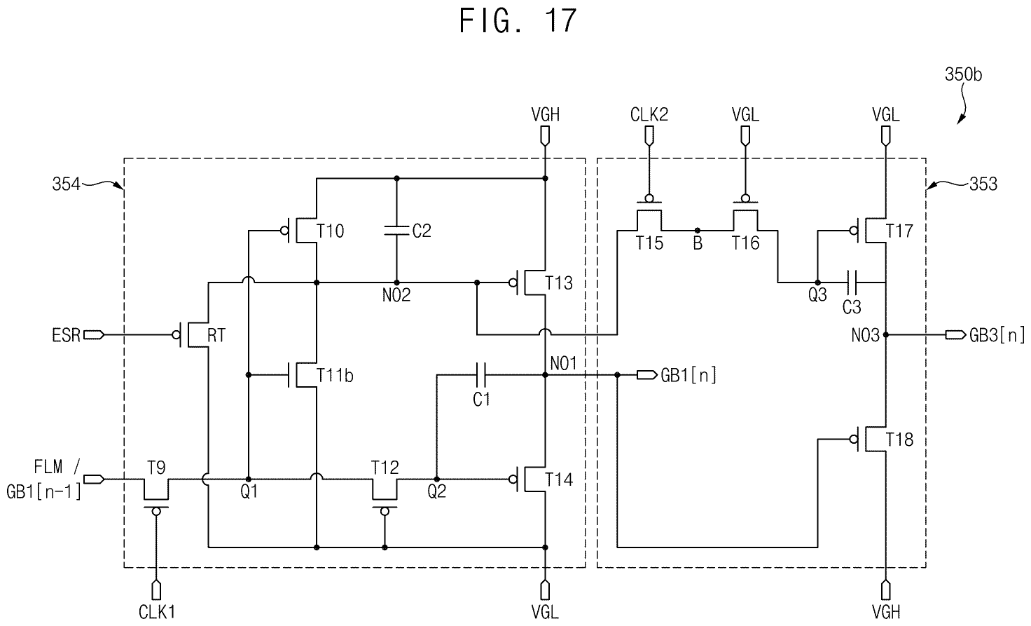

FIG. 17 is a circuit diagram illustrating a gate driving circuit 350b of a display device according to an embodiment of the present inventive concept.

The gate driving circuit 350b according to the present embodiment is substantially the same as the gate driving circuit 350a of the previous embodiment explained referring to FIG. 16 except that the gate driving circuit 350b further include a reset transistor RT. Thus, the same reference numerals will be used to refer to the same or like parts as those described in the previous embodiment of FIG. 16 and any repetitive explanation concerning the above elements will be omitted.

Referring to FIGS. 1, 4 to 6, 16 and 17, the gate driving circuit 350b may further include the reset transistor RT. The reset transistor RT may include a control electrode receiving a reset signal ESR, a first electrode connected to the second output node NO2 and second electrode receiving the second gate power supply voltage VGL.

When the reset signal ESR has the low voltage VGL, the reset transistor RT may be turned on. The reset transistor RT may transfer the second gate power supply voltage VGL to the second output node NO2. When the second output node NO2 has the low voltage VGL, the thirteenth transistor T13 may be turned on. The thirteenth transistor T13 may transfer the first gate power supply voltage VGH to the first output node NO1. The first output node NO1 may have the high voltage VGH.

When the display device is first driven, the reset transistor RT may reset the voltage of the output node NO2 to the low voltage VGL. Accordingly, the stability and reliability of the gate driving circuit 350b may be increased.

When the first gate signal GB1[n] and the third gate signal GB3[n], which have opposite phases, are output simultaneously from the gate driving circuit 350b to the pixel circuit, a separate gate driving circuit is not required. Accordingly, the number of gate driving circuits in the display device may be reduced, and the dead space of the display device may be reduced.

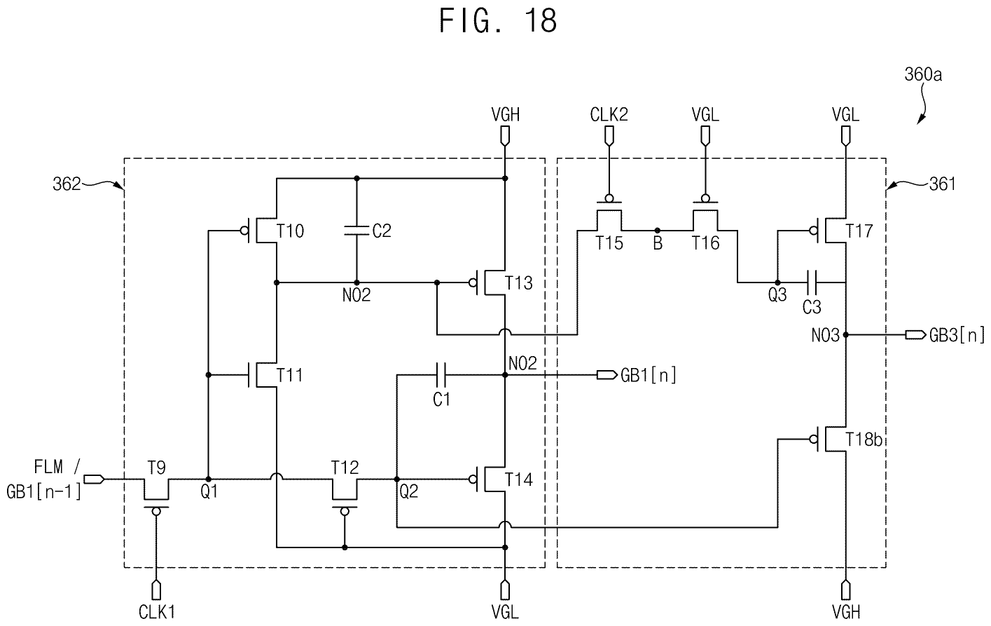

FIG. 18 is a circuit diagram illustrating a gate driving circuit 360a of a display device according to an embodiment of the present inventive concept.

The gate driving circuit 360a according to the present embodiment is substantially the same as the gate driving circuit 350a of the previous embodiment explained referring to FIG. 16 except that a control electrode of an eighteenth transistor T18b is connected to the second node Q2. Thus, the same reference numerals will be used to refer to the same or like parts as those described in the previous embodiment of FIG. 16 and any repetitive explanation concerning the above elements will be omitted.

Referring to FIGS. 1, 4 to 6, 16 and 18, the eighteenth transistor T18b of the gate driving circuit 360a may include the control electrode connected to the second node Q2, the first electrode connected to the third output node NO3 and the second electrode receiving the first gate power supply voltage VGH.

When the first gate signal GB1[n] and the third gate signal GB3[n], which have opposite phases, are output simultaneously from the gate driving circuit 360a to the pixel circuit, a separate gate driving circuit is not required. Accordingly, the number of gate driving circuits in the display device may be reduced, and the dead space of the display device may be reduced.

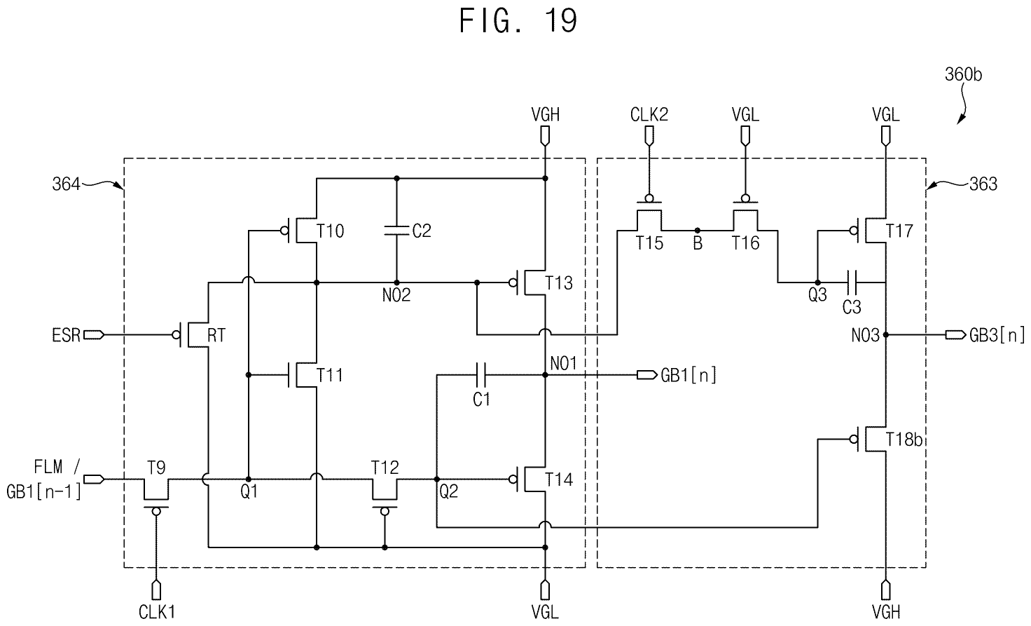

FIG. 19 is a circuit diagram illustrating a gate driving circuit 360b of a display device according to an embodiment of the present inventive concept.

The gate driving circuit 360b according to the present embodiment is substantially the same as the gate driving circuit 360a of the previous embodiment explained referring to FIG. 18 except that the gate driving circuit 360b further include a reset transistor RT. Thus, the same reference numerals will be used to refer to the same or like parts as those described in the previous embodiment of FIG. 18 and any repetitive explanation concerning the above elements will be omitted.

Referring to FIGS. 1, 4 to 6, 16, 18 and 19, the gate driving circuit 360b may further include the reset transistor RT.

The reset transistor RT may include a control electrode receiving a reset signal ESR, a first electrode connected to the second output node NO2 and second electrode receiving the second gate power supply voltage VGL.

When the reset signal ESR has the low voltage VGL, the reset transistor RT may be turned on. The reset transistor RT may transfer the second gate power supply voltage VGL to the second output node NO2. When the second output node NO2 has the low voltage VGL, the thirteenth transistor T13 may be turned on. The thirteenth transistor T13 may transfer the first gate power supply voltage VGH to the first output node NO1. The first output node NO1 may have the high voltage VGH.

When the display device is first driven, the reset transistor RT may reset the voltage of the output node NO2 to the low voltage VGL. Accordingly, the stability and reliability of the gate driving circuit 360b may be increased.

When the first gate signal GB1[n] and the third gate signal GB3[n], which have opposite phases, are output simultaneously from the gate driving circuit 360b to the pixel circuit, a separate gate driving circuit is not required. Accordingly, the number of gate driving circuits in the display device may be reduced, and the dead space of the display device may be reduced.

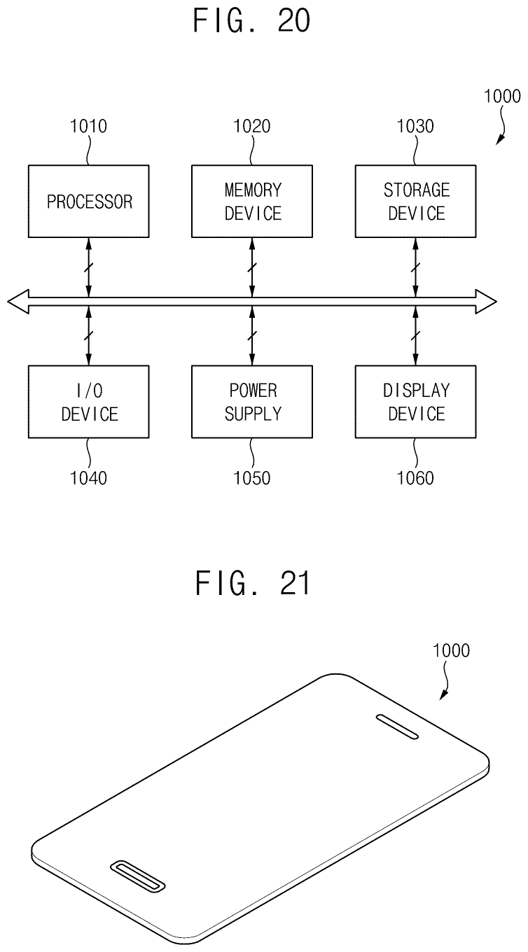

FIG. 20 is a block diagram illustrating an electronic device 1000. FIG. 21 is a diagram illustrating an embodiment in which the electronic device 1000 of FIG. 20 is implemented as a smart phone.

Referring to FIGS. 20 and 21, the electronic device 1000 may include a processor 1010, a memory device 1020, a storage device 1030, an input/output (I/O) device 1040, a power supply 1050, and a display device 1060. The display device 1060 may be the display device of FIG. 1. In addition, the an electronic device 1000 may further include a plurality of ports for communicating with a video card, a sound card, a memory card, a universal serial bus (USB) device, other electronic device, and the like.

In an embodiment, as illustrated in FIG. 21, the an electronic device 1000 may be implemented as a smart phone. However, the an electronic device 1000 is not limited thereto. For example, the an electronic device 1000 may be implemented as a cellular phone, a video phone, a smart pad, a smart watch, a tablet PC, a car navigation system, a computer monitor, a laptop, a head mounted display (HMD) device, and the like.

The processor 1010 may perform various computing functions. The processor 1010 may be a micro processor, a central processing unit (CPU), an application processor (AP), and the like. The processor 1010 may be coupled to other components via an address bus, a control bus, a data bus, and the like. Further, the processor 1010 may be coupled to an extended bus such as a peripheral component interconnection (PCI) bus.

The memory device 1020 may store data for operations of the an electronic device 1000. For example, the memory device 1020 may include at least one non-volatile memory device such as an erasable programmable read-only memory (EPROM) device, an electrically erasable programmable read-only memory (EEPROM) device, a flash memory device, a phase change random access memory (PRAM) device, a resistance random access memory (RRAM) device, a nano floating gate memory (NFGM) device, a polymer random access memory (PoRAM) device, a magnetic random access memory (MRAM) device, a ferroelectric random access memory (FRAM) device, and the like and/or at least one volatile memory device such as a dynamic random access memory (DRAM) device, a static random access memory (SRAM) device, a mobile DRAM device, and the like.

The storage device 1030 may include a solid state drive (SSD) device, a hard disk drive (HDD) device, a CD-ROM device, and the like.

The I/O device 1040 may include an input device such as a keyboard, a keypad, a mouse device, a touch-pad, a touch-screen, and the like, and an output device such as a printer, a speaker, and the like. In some embodiments, the I/O device 1040 may include the display device 1060.

The power supply 1050 may provide power for operations of the an electronic device 1000. For example, the power supply 1050 may be power management integrated circuit (PMIC).

The display device 1060 may display am image corresponding to visual information of the an electronic device 1000. At this time, the display device 1060 may be an organic light emitting display device (OLED) or quantum dot light emitting display device (QLED). However, the display device 1060 is not limited thereto. The display device 1060 may be connected to other components through buses or other communication links.

In an embodiment, the display device 1060 may include the display panel 100 including the pixel circuit PX1 including transistors having different types of transistors (N-type or P-type). The transistors having different types of transistors may receive two gate signals having opposite phases with each other. In addition, the display device 1060 may include the gate driving circuit 300. The gate driving circuit 300 may output two gate signals having opposite phases to the pixel circuit PX1. However, the connection relationship and operation of the pixel circuit PX1 and the gate driving circuit 300 are described in FIGS. 1 to 19, any repetitive explanation concerning the above elements will be omitted.

The inventive concepts may be applied to any display device and any electronic device including the touch panel. For example, the inventive concepts may be applied to a mobile phone, a smart phone, a tablet computer, a digital television (TV), a 3D TV, a personal computer (PC), a home appliance, a laptop computer, a personal digital assistant (PDA), a portable multimedia player (PMP), a digital camera, a music player, a portable game console, a navigation device, etc.

The foregoing is illustrative of the inventive concept and is not to be construed as limiting thereof. Although a few embodiments of the inventive concept have been described, those skilled in the art will readily appreciate that many modifications are possible in the embodiments without materially departing from the novel teachings and advantages of the inventive concept. Accordingly, all such modifications are intended to be included within the scope of the inventive concept as defined in the claims. In the claims, means-plus-function clauses are intended to cover the structures described herein as performing the recited function and not only structural equivalents but also equivalent structures. Therefore, it is to be understood that the foregoing is illustrative of the inventive concept and is not to be construed as limited to the specific embodiments disclosed, and that modifications to the disclosed embodiments, as well as other embodiments, are intended to be included within the scope of the appended claims. The inventive concept is defined by the following claims, with equivalents of the claims to be included therein.

What is claimed is:

1. A gate driving circuit comprising:
a normal output circuit configured to output a first gate signal at a first output node and a second gate signal at a second output node in response to a previous first gate signal and a first clock signal,
wherein a phase of the first gate signal is opposite to a phase of the second gate signal, and
wherein the first gate signal and the second gate signal are applied to different transistors included in a pixel circuit.

2. The gate driving circuit of claim 1, wherein the normal output circuit includes:
a ninth transistor including a control electrode configured to receive the first clock signal, a first electrode configured to receive the previous first gate signal and a second electrode connected to a first node;
a tenth transistor including a control electrode connected to the first node, a first electrode configured to receive a first gate power supply voltage and a second electrode connected to the second output node;
an eleventh transistor including a first electrode connected to the second output node and a second electrode configured to receive a second gate power supply voltage;
a twelfth transistor including a control electrode configured to receive the second gate power supply voltage, a first electrode connected to the first node and a second electrode connected to a second node;
a thirteenth transistor including a control electrode connected to the second output node, a first electrode configured to receive the first gate power supply voltage and a second electrode connected to the first output node;
a fourteenth transistor including a control electrode connected to the second node, a first electrode connected to the first output node and a second electrode configured to receive the second gate power supply voltage;
a first capacitor including a first electrode connected to the second node and a second electrode connected to the first output node; and
a second capacitor including a first electrode configured to receive the first gate power supply voltage and a second electrode connected to the second output node.

3. The gate driving circuit of claim 2, wherein the eleventh transistor further includes a control electrode connected to the second node.

4. The gate driving circuit of claim 2, wherein the eleventh transistor further includes a control electrode connected to the first node.

5. The gate driving circuit of claim 2, wherein the normal output circuit further includes a reset transistor including a control electrode configured to receive a reset signal, a first electrode connected to the second output node and a second electrode configured to receive the second gate power supply voltage.

6. A gate driving circuit comprising:
a normal output circuit configured to output a first gate signal at a first output node and control a voltage of a second output node in response to a previous first gate signal and a first clock signal; and
an inverted output circuit configured to output a third gate signal at a third output node in response to a second clock signal and the voltage of the second output node,
wherein a phase of the first gate signal is opposite to a phase of the third gate signal, and
wherein the first gate signal and the third gate signal are applied to different transistors included in a pixel circuit.

7. The gate driving circuit of claim 6, wherein the inverted output circuit includes:
a fifteenth transistor including a control electrode configured to receive the second clock signal, a first electrode connected to the second output node and a second electrode connected to a buffer node;
a sixteenth transistor including a control electrode configured to receive a second gate power supply voltage, a first electrode connected to the buffer node and a second electrode connected to a third node;
a seventeenth transistor including a control electrode connected to the third node, a first electrode configured to receive the second gate power supply voltage and a second electrode connected to the third output node;
an eighteenth transistor including a first electrode connected to the third output node and a second electrode configured to receive a first gate power supply voltage; and
a third capacitor including a first electrode connected to the third node and a second electrode connected to the third output node.

8. The gate driving circuit of claim 7, wherein the normal output circuit includes:
a ninth transistor including a control electrode configured to receive the first clock signal, a first electrode configured to receive the previous first gate signal and a second electrode connected to a first node;
a tenth transistor including a control electrode connected to the first node, a first electrode configured to receive the first gate power supply voltage and a second electrode connected to the second output node;
an eleventh transistor including a first electrode connected to the second output node and a second electrode configured to receive the second gate power supply voltage;
a twelfth transistor including a control electrode configured to receive the second gate power supply voltage, a first electrode connected to the first node and a second electrode connected to a second node;
a thirteenth transistor including a control electrode connected to the second output node, a first electrode configured to receive the first gate power supply voltage and a second electrode connected to the first output node;
a fourteenth transistor including a control electrode connected to the second node, a first electrode connected to the first output node and a second electrode configured to receive the second gate power supply voltage;
a first capacitor including a first electrode connected to the second node and a second electrode connected to the first output node; and
a second capacitor including a first electrode configured to receive the first gate power supply voltage and a second electrode connected to the second output node.

9. The gate driving circuit of claim 8, wherein the eighteenth transistor further includes a control electrode connected to the first output node.

10. The gate driving circuit of claim 8, wherein the eighteenth transistor further includes a control electrode connected to the second node.

11. The gate driving circuit of claim 8, wherein the normal output circuit further includes a reset transistor including a control electrode configured to receive a reset signal, a first electrode connected to the second output node and a second electrode configured to receive the second gate power supply voltage.

12. A display device comprising:
a display panel including a pixel circuit;
a gate driving circuit configured to output a gate signal to the pixel circuit;
a data driving circuit configured to output a data voltage to the pixel circuit; and
an emission driving circuit configured to output an emission signal to the pixel circuit,
wherein the pixel circuit includes:
a light emitting element;
a first transistor configured to apply a driving current to the light emitting element;
a second transistor including a first electrode configured to receive a bias voltage and a second electrode connected to a first electrode of the first transistor; and
a third transistor including a first electrode connected to an anode electrode of the light emitting element and a second electrode configured to receive a first initialization voltage; and
wherein one of the second transistor and the third transistor is a P-type transistor and an other one of the second transistor and the third transistor is an N-type transistor.

13. The display device of claim 12, wherein the second transistor is the P-type transistor and the third transistor is the N-type transistor.

14. The display device of claim 13, wherein the pixel circuit further includes:
a fourth transistor including a control electrode configured to receive a writing gate signal, a first electrode configured to receive the data voltage and a second electrode connected to the first electrode of the first transistor;
a fifth transistor including a control electrode configured to receive a compensation gate signal, a first electrode connected to a control electrode of the first transistor and a second electrode connected to a second electrode of the first transistor;
a sixth transistor including a control electrode configured to receive an initialization gate signal, a first electrode connected to the control electrode of the first transistor and a second electrode configured to receive a second initialization voltage;
a seventh transistor including a control electrode configured to receive the emission signal, a first electrode configured to receive a first power voltage and a second electrode connected to the first electrode of the first transistor;
an eighth transistor including a control electrode configured to receive the emission signal, a first electrode connected to the second electrode of the first transistor and a second electrode connected to the first electrode of the third transistor; and
a storage capacitor including a first electrode configured to receive the first power voltage and a second electrode connected to the control electrode of the first transistor.

15. The display device of claim 14, wherein the third transistor further includes a control electrode and a second control electrode connected to the control electrode of the third transistor,
wherein the fifth transistor further includes a second control electrode connected to the control electrode of the fifth transistor, and
wherein the sixth transistor further includes a second control electrode connected to the control electrode of the sixth transistor.

16. The display device of claim 14, wherein the first transistor further includes a second control electrode configured to receive the first power voltage.

17. The display device of claim 13, wherein the gate driving circuit includes a normal output circuit configured to output a first gate signal at a first output node and a second gate signal at a second output node in response to a previous first gate signal and a first clock signal,
wherein a phase of the first gate signal is opposite to a phase of the second gate signal, and
wherein the first gate signal and the second gate signal are applied to different transistors included in the pixel circuit.

18. The display device of claim 17, wherein the second transistor further includes a control electrode configured to receive the first gate signal, and
wherein the third transistor further includes a control electrode configured to receive the second gate signal.

19. The display device of claim 13, wherein the gate driving circuit includes:
a normal output circuit configured to output a first gate signal at a first output node and control a voltage of a second output node in response to a previous first gate signal and a first clock signal; and
an inverted output circuit configured to output a third gate signal at a third output node in response to a second clock signal and the voltage of the second output node,
wherein a phase of the first gate signal is opposite to a phase of the third gate signal, and
wherein the first gate signal and the third gate signal are applied to different transistors included in the pixel circuit.

20. The display device of claim 19, wherein the second transistor further includes a control electrode configured to receive the first gate signal, and
wherein the third transistor further includes a control electrode configured to receive the third gate signal.

21. An electronic device comprising:
a display panel including a pixel circuit;
a gate driving circuit configured to output a gate signal to the pixel circuit;
a data driving circuit configured to output a data voltage to the pixel circuit;
an emission driving circuit configured to output an emission signal to the pixel circuit;
a driving control circuit configured to control the gate driving circuit, the data driving circuit and the emission driving circuit based on an input control signal and an input image data; and
a processor configured to output the input control signal and the input image data to the driving control circuit,
wherein the pixel circuit includes:
a light emitting element;
a first transistor configured to apply a driving current to the light emitting element;
a second transistor including a first electrode configured to receive a bias voltage and a second electrode connected to a first electrode of the first transistor; and
a third transistor including a first electrode connected to an anode electrode of the light emitting element and a second electrode configured to receive a first initialization voltage; and
wherein one of the second transistor and the third transistor is a P-type transistor and an other one of the second transistor and the third transistor is an N-type transistor.
